(19) United States
(12) Patent Application Publication
(10) Pub. No.: US 2026/0014228 A1
(43) Pub. Date: Jan. 15, 2026

(54) COMPOSITION AND USE METHOD THEREFOR

(71) Applicant: NANJING REJU THERAPEUTICS, INC., Nanjing, Jiangsu (CN)

(72) Inventors: Yin JI, Nanjing, Jiangsu (CN); Xia LIU, Nanjing, Jiangsu (CN); Yao WU, Nanjing, Jiangsu (CN)

(21) Appl. No.: 18/992,879

(22) PCT Filed: Jul. 12, 2023
(86) PCT No.: PCT/CN2023/107077

§ 371 Date: Jan. 9, 2025

(30) Foreign Application Priority Data
Jul. 12, 2022 (CN) 202210815980.5

(51) Int. Cl. A61K 38/17; C07K 14/435

(52) U.S. Cl. CPC A61K 38/17; C07K 14/435

(57) ABSTRACT

The present invention provides a composition based on three factors and used for restoring viability of cells, tissue, or organs of a subject, and a method for using the composition to restore viability of the cells, tissue, or organs of the subject. By means of the composition and the method, the viability or functions of the cells, tissue, or organs of the subject can be restored. The composition and the method can be used for any disease or damage that requires repairing or replacement of malfunctional tissue.

### BACKGROUND OF THE INVENTION

All organisms undergo aging accompanied by a decline in the functional viability of cells and tissues. Aging is characterized by a gradual loss of function at the molecular, cellular, tissue and organismal levels. The main characteristics include problems with telomere attrition, genetic instability, epigenetic and transcriptional alterations, and the accumulation of error-prone proteins. In many animals, including vertebrates, the ability to regenerate and repair vital organs declines dramatically with the onset of aging and disease. Unlike adult somatic cells, individual cells that are chronologically closer to the time of fertilization (such as embryonic and infant cells) exhibit a youthful state of cells and possess a greater ability to resist damage and stress, and are capable of healing, renewing, and regenerating organs and tissues. Accordingly, compositions and methods for rejuvenating viability of the cells and thereby restoring them from an aged, mature state to a younger, more vigorous state of life have long been sought for treating certain injuries and diseases, as well as for overall reversing and preventing aging in whole organisms.

In 2006, Professor Shinya Yamanaka of Kyoto University in Japan developed induced pluripotent stem cell (iPSC) technology that reverses telomere attrition and oxidative stress in cells. This milestone cell technology, in brief, involves reprogramming cells with four transcription factors (Oct4, Sox2, Klf4, and c-Myc). A combination of the four transcription factors (Oct4, Sox2, Klf4, and c-Myc) is introduced into differentiated somatic cells with a viral vector to reprogram these cells to obtain a cell that closely resembles embryonic stem cells. Thus, this reverts the somatic cells to a state similar to that of embryonic stem cells. These cells are similar to human embryonic stem cells and have exceptional differentiation potential. They can differentiate into various human cell types, such as blood cells, bone cells, and nerve cells, which can then be cultured to produce human organs, bones, comeas, pancreas, etc. It has great potential for applications in disease modeling, drug screening, and cell therapy.

After 2016, scientists discovered that four transcription factors (Oct4, Sox2, Klf4, and c-My) can also reprogram senescent cells into younger cells. However, the existing combinations of reprogramming factors that can achieve anti-aging or restoration of cell viability are relatively limited, and their safety requires further improvement. Therefore, there is an urgent need in the field for more and safer reprogramming factors that can rejuvenate a cell, tissue or organ of a subject.

### SUMMARY OF THE INVENTION

The present invention relates to a composition for restoring viability of a cell, tissue or organ of a subject, and a method for using the composition to restore viability of the cell, tissue or organ of the subject. By means of the composition and the method, the viability or functions of the cell, tissue or organ of the subject can be restored. The composition and the method can be used for any disease or damage that requires repairing or replacement of malfunctional tissue.

In a first aspect, the present invention provides a composition for restoring viability of a cell, tissue or organ of a subject, which comprises a factor selected from the group consisting of SOX1, SOX2, SOX3, and GMNN, a factor selected from the group consisting of GATA3 and GATA6, and a factor of Klf4.

In some embodiments, the composition comprises GATA6, SOX2 and Klf4.

In some embodiments, the composition comprises GATA6, SOX1 and Klf4.

In some embodiments, the composition comprises GATA3, SOX2 and Klf4.

In some embodiments, the composition comprises GATA3, SOX1 and Klf4.

In some embodiments, the composition comprises GATA6, SOX3 and Klf4.

In some embodiments, the composition comprises GATA6, GMNN and Klf4.

In some embodiments, the composition comprises GATA3, SOX3 and Klf4.

In some embodiments, the composition comprises GATA3, GMNN and Klf4.

In some embodiments, any one of the factors is a protein, a nucleic acid encoding said protein, or a mixture thereof.

For example, any one of the factors of SOX1, SOX2, SOX3, GMNN, GATA3, GATA6, and Klf4 can be provided as a protein, a fusion protein with a transmembrane domain, or a nucleic acid encoding the same, such as in a form of DNA or RNA.

In some embodiments, any one of the factors is provided in a form of an expression vector. The expression vector can be a plasmid vector, an episomal vector, a transposon, a virus-derived vector such as an adenovirus vector, an adeno-associated virus vector (AAV), a lentivirus vector, a Sendai virus vector, a cytomegalovirus vector, or a vaccinia virus vector.

In some embodiments, the factors are present on one or more expression vectors.

In some embodiments, the factors are present on a same expression vector.

In some embodiments, any one of the factors is provided in a form of mRNA.

In some embodiments, the factors are in tandem on a same mRNA molecule, or each factor is on a different mRNA molecule, thereby the factors being in the form of a combination of multiple mRNA molecules.

In some embodiments, any one of the factors is a synthesized mRNA, which encodes a wild-type form, a mutant, or a genetically engineered form of any one of SOX1, SOX2, SOX3, GMNN, GATA3, GATA6, and Klf4.

In some embodiments, the composition further comprises a pharmaceutically acceptable carrier or adjuvant.

In some embodiments, the subject is a mammal, and the mammal includes, but is not limited to, a human, a mouse, a rat, a cattle, a sheep, a horse, a canine, a cat, a pig, or a monkey.

In some embodiments, the subject is a chicken, a duck, a goose or a bird.

In some embodiments, the cell, tissue or organ of the subject is derived from, but not limited to, eye, ear, nose, mouth including gum and tooth root; bone, lung, breast, pancreas, stomach, esophagus; muscle, including cardiac muscle; liver, blood vessel; skin, including hair; heart, brain, neural tissue, kidney, testis, prostate, penis, cloaca, fin, ovary, or intestine.

In some embodiments, the composition for restoring viability of a cell, tissue or organ of a subject can restore an epigenetic information of the cell, tissue or organ of the subject.

In some embodiments, the composition for restoring viability of a cell, tissue or organ of a subject can restore an epigenetic information lost due to aging, damage, or disease in the cell, tissue or organ of the subject.

In some embodiments, the expression of any one of the factors can be regulated by a promoter to reduce and reverse epigenetic marks associated with aging, increase epigenetic marks associated with cellular rejuvenation, reduce expression of proteins associated with aging, increase expression of proteins associated with cellular rejuvenation, restore a balance between euchromatin and heterochromatin, prevent loss of cellular identity, restore cellular identity, and reverse DNA methylation changes associated with aging, thereby rejuvenating cells.

In some embodiments, any one of the factors of SOX1, SOX2, SOX3, GMNN, GATA3, GATA6, and Klf4 is a human protein, or non-human protein, such as a protein of a mammal (e.g., a mouse, a rat, a cattle, a sheep, a horse, a canine, a cat, a pig, a monkey, a chicken, a duck, a goose, a bird) or a protein having at least 80% identity to the amino acid sequence of the corresponding human or non-human protein, and maintaining the activity

In some embodiments, any one of the factors of SOX1, SOX2, SOX3, GMNN, GATA3, GATA6, and Klf4 or any combination thereof is a nucleic acid encoding a human protein, or a nucleic acid encoding a non-human protein, such as a nucleic acid encoding a protein of a mammal (e.g., a mouse, a rat, a cattle, a sheep, a horse, a canine, a cat, a pig, a monkey, a chicken, a duck, a goose, a bird), or a nucleic acid having at least 80% identity to the corresponding nucleic acid sequence encoding the human or non-human protein, and maintaining the activity.

In some embodiments, restoring viability of a cell, tissue or organ of a subject includes restoring transcriptional profile or an epigenetic information lost in at least one cell, tissue, or organ due to aging, damage, disease, or any combination thereof.

In some embodiments, restoring viability of a cell, tissue or organ of a subject includes restoring a function of the cell, increasing a potential of the cell, enhancing a viability of the cell, or increasing a replicative capacity or lifespan of the cell, or a combination thereof.

In some embodiments, restoring viability of a cell, tissue or organ of a subject includes promoting axonal regeneration of a neuronal cell in a subject; proliferating skin fibroblasts in a subject; promoting proliferation of chondrocytes in a subject; promoting proliferation of muscle stem cells in a subject; reversing aging of skin fibroblasts in a subject; or reversing aging of chondrocytes.

In some embodiments, the subject suffers from, is suspected to suffer from, or is at risk of suffering from:
a disease, condition, or symptom related to impaired viability of the cell, tissue or organ, or aging;
the disease, condition, or symptom related to impaired viability of the cell, tissue or organ, or aging is, for example, a neurodegenerative disease, a cardiovascular disease, a metabolic disease, a musculoskeletal disease, an inflammatory disease, a symptom or disorder associated with a skin, an ocular disease, or a disease associated with the reproductive system;
the neurodegenerative disease is stroke, Huntington's Disease, dementia, Alzheimer's Disease, or Parkinson's Disease;
the cardiovascular disease is hypertension, arrhythmia of coronary heart disease, heart disease, hypotension, heart failure, angina pectoris, arteriosclerosis, myocardial infarction, myocarditis, congestive heart failure, atrioventricular block, atherosclerosis, or myocardial infarction;
the metabolic disease is type I diabetes, type II diabetes, hyperglycemia, hyperinsulinemia, insulin resistance, obesity, or gout;
the musculoskeletal disease is muscular dystrophy, amyosthenia, myasthenia gravis, osteoporosis, osteoarthritis, osteochondrosis, osteochondritis, osteonecrosis, or osteopenia;

the inflammatory disease is systemic lupus erythematosus, polymyalgia rheumatica, gouty arthritis, degenerative arthritis, rheumatoid arthritis, inflammatory arthritis, Hashimoto's thyroiditis, inflammatory bowel disease, hepatitis, pneumonia, respiratory inflammation, or encephalitis;
the aging symptom or disorder associated with skin is wrinkle, senile plaque, seborrheic keratosis, tinea manus and pedis, urticaria, neurodermatitis, or pigmented nevus;
the ocular disease is macular degeneration, retinal degeneration, retinal detachment, diabetic retinopathy, glaucoma, optic atrophy, floaters, cataract, non-arteritic anterior ischemic optic neuropathy, chorioretinitis, retinitis pigmentosa, traumatic optic neuropathy, or compressive optic neuropathy; and
the disease associated with the reproductive system is erectile dysfunction or premature ovarian failure.

In some embodiments, the composition can induce cell reprogramming, reverse aging, improve tissue function, improve organ function, promote tissue repair, promote tissue survival, promote tissue regeneration, promote tissue growth, promote angiogenesis, reduce scar formation, alleviate external manifestations of aging including alopecia, scant hair, graying of hair, anetoderma, and wrinkle of skin, promote regeneration of organs, promote organ survival, treat disease or any combination thereof.

In some embodiments, the composition can reverse aging of a cell, tissue, or organ of a subject, thereby restoring viability of the cell, tissue, or organ.

In some embodiments, the composition can promote neuronal regeneration in a subject.

In some embodiments, the composition can proliferate skin fibroblasts in a subject.

In some embodiments, the composition can promote proliferation of chondrocytes in a subject.

In some embodiments, the composition can promote proliferation of muscle stem cells in a subject.

In some embodiments, the method can reverse aging of skin fibroblasts in a subject.

In some embodiments, the composition can reverse aging of chondrocytes.

In some embodiments, the composition does not induce formation of teratoma or does not induce growth of tumor or formation of tumor.

In some embodiments, the composition does not induce complete reprogramming.

In some embodiments, the composition does not reprogram a cell into an induced pluripotent stem cell (ipsc).

In a second aspect, the present invention provides a method for restoring viability of a cell, tissue or organ of a subject, and the method comprises administering a composition of the first aspect to a subject in vivo.

The composition comprises a factor selected from the group consisting of SOX1, SOX2, SOX3, and GMNN, a factor selected from the group consisting of GATA3 and GATA6, and a factor of Klf4.

In some embodiments, the composition comprises GATA6, SOX2 and Klf4.

In some embodiments, the composition comprises GATA6, SOX1 and Klf4.

In some embodiments, the composition comprises GATA3, SOX2 and Klf4.

In some embodiments, the composition comprises GATA3, SOX1 and Klf4.

In some embodiments, the composition comprises GATA6, SOX3 and Klf4.

In some embodiments, the composition comprises GATA6, GMNN and Klf4.

In some embodiments, the composition comprises GATA3, SOX3 and Klf4.

In some embodiments, the composition comprises GATA3, GMNN and Klf4.

In some embodiments, any one of the factors is a protein, a nucleic acid encoding said proteins, or a mixture thereof.

For example, any one of the factors of SOX1, SOX2, SOX3, GMNN, GATA3, GATA6, and Klf4 can be provided as a protein, a fusion protein with a transmembrane domain, or a nucleic acid encoding the same, such as in a form of DNA or RNA.

In some embodiments, any one of the factors is provided in a form of an expression vector. The expression vector can be a plasmid vector, an episomal vector, a transposon, a virus-derived vector such as an adenovirus vector, an adeno-associated virus vector (AAV), a lentivirus vector, a Sendai virus vector, a cytomegalovirus vector, or a cowpox virus vector.

In some embodiments, the factors are present on one or more expression vectors.

In some embodiments, the factors are present on a same expression vector.

In some embodiments, any one of the factors is provided in a form of mRNA.

In some embodiments, the factors are in tandem on a same mRNA molecule, or each factor is on a different mRNA molecule, thereby the factors being in the form of a combination of multiple mRNA molecules.

In some embodiments, any one of the factors is a synthesized mRNA, which encodes a wild-type form, a mutant, or a genetically engineered form of any one of SOX1, SOX2, SOX3, GMNN, GATA3, GATA6, and Klf4.

In some embodiments, the composition further comprises a pharmaceutically acceptable carrier or adjuvant.

In some embodiments, the subject is a mammal, and the mammal includes, but is not limited to, a human, a mouse, a rat, a cattle, a sheep, a horse, a canine, a cat, a pig, or a monkey.

In some embodiments, the subject is a chicken, a duck, a goose or a bird.

In some embodiments, the cell, tissue or organ of the subject is derived from, but not limited to, eye, ear, nose, mouth including gum and tooth root; bone, Jung, breast, pancreas, stomach, esophagus; muscle, including cardiac muscle; liver, blood vessel; skin, including hair; heart, brain, neural tissue, kidney, testis, prostate, penis, cloaca, fin, ovary, or intestine.

In some embodiments, the composition for restoring viability of a cell, tissue or organ of a subject can restore an epigenetic information of the cell, tissue or organ of the subject.

In some embodiments, the composition for restoring viability of a cell, tissue or organ of a subject can restore an epigenetic information lost due to aging, damage, or disease in the cell, tissue or organ of the subject

In some embodiments, the expression of any one of the factor can be regulated by a promoter to reduce and reverse epigenetic marks associated with aging, increase epigenetic marks associated with cellular rejuvenation, reduce expression of proteins associated with aging, increase expression of proteins associated with cellular rejuvenation, restore a balance between euchromatin and heterochromatin, prevent loss of cellular identity, restore cellular identity, and reverse DNA methylation changes associated with aging, thereby restoring viability of cells.

In some embodiments, any one of the factors of SOX1, SOX2, SOX3, GMNN, GATA3, GATA6, and Klf4 is a human protein, or a non-human protein, such as a protein of a mammal (e.g., a mouse, a rat, a cattle, a sheep, a horse, a canine, a cat, a pig, a monkey, a chicken, a duck, a goose, a bird) or a protein having at least 80% identity to the amino acid sequence of the corresponding human or non-human protein, and maintaining the activity.

In some embodiments, any one of the factors of SOX1, SOX2, SOX3, GMNN, GATA3, GATA6, and Klf4 or any combination thereof is a nucleic acid encoding a human protein, or a nucleic acid encoding a non-human protein, such as a nucleic acid encoding a protein of a mammal (e.g., a mouse, a rat, a cattle, a sheep, a horse, a canine, a cat, a pig, a monkey, a chicken, a duck, a goose, a bird), or a nucleic acid having at least 80% identity to the corresponding nucleic acid sequence encoding the human or non-human protein, and maintaining the activity.

In a third aspect, the present invention provides a use of the composition of the first aspect for the preparation of a medicament for restoring viability of a cell, tissue or organ of the subject.

In some embodiments, restoring viability of cell, tissue or organ of a subject includes restoring transcriptional profile or an epigenetic information lost in at least one cell, tissue, or organ due to aging, damage, disease, or any combination thereof.

In some embodiments, restoring viability of cell, tissue or organ of a subject includes restoring a function of the cell, increasing a potential of the cell, enhancing a viability of the cell, or increasing a replicative capacity or lifespan of the cell, or a combination thereof.

In some embodiments, restoring viability of cell, tissue or organ of a subject includes promoting axonal regeneration of a neuronal cell in a subject; proliferating skin fibroblasts in a subject; promoting proliferation of chondrocytes in a subject; promoting proliferation of muscle stem cells in a subject; reversing aging of skin fibroblasts in a subject; or reversing aging of chondrocytes.

In some embodiments, the subject suffers from, is suspected to suffer from, or is at risk of suffering from:
a disease, condition, or symptom related to impaired viability of the cell, tissue or organ, or aging;

the disease, condition, or symptom related to impaired viability of the cell, tissue or organ, or aging is, for example, a neurodegenerative disease, a cardiovascular disease, a metabolic disease, a musculoskeletal disease, an inflammatory disease, a symptom or disorder associated with a skin, an ocular disease, or a disease associated with the reproductive system;
the neurodegenerative disease is stroke. Huntington's Disease, dementia, Alzheimer's Disease, or Parkinson's Disease;

the cardiovascular disease is hypertension, arrhythmia of coronary heart disease, heart disease, hypotension, heart failure, angina pectoris, arteriosclerosis, myocardial infarction, myocarditis, congestive heart failure, atrioventricular block, atherosclerosis, or myocardial infarction;
the metabolic disease is type I diabetes, type II diabetes, hyperglycemia, hyperinsulinemia, insulin resistance, obesity, or gout;
the musculoskeletal disease is muscular dystrophy, amyosthenia, myasthenia gravis. osteoporosis, osteoarthritis, osteochondrosis, osteochondritis, osteonecrosis, or osteopenia;
the inflammatory disease is systemic lupus erythematosus, polymyalgia rheumatica, gouty arthritis, degenerative arthritis, rheumatoid arthritis, inflammatory arthritis, Hashimoto's thyroiditis, inflammatory bowel disease, hepatitis, pneumonia, respiratory inflammation, or encephalitis;
the aging symptom or disorder associated with skin is wrinkle, senile plaque, seborrheic keratosis, tinea manus and pedis, urticaria, neurodermatitis, or pigmented nevus;
the ocular disease is macular degeneration, retinal degeneration, retinal detachment, diabetic retinopathy, glaucoma, optic atrophy, floaters, cataract, non-arteritic anterior ischemic optic neuropathy, chorioretinitis, retinitis pigmentosa, traumatic optic neuropathy, or compressive optic neuropathy; and
the disease associated with the reproductive system is erectile dysfunction or premature ovarian failure.

In some embodiments, the method can induce cell reprogramming, reverse aging, improve tissue function, improve organ function, promote tissue repair, promote tissue survival, promote tissue regeneration, promote tissue growth, promote angiogenesis, reduce scar formation, alleviate external manifestations of aging including alopecia, scant hair, graving of hair, anetoderma, and wrinkle of skin, promote regeneration of organs, promote organ survival, treat disease or any combination thereof.

In some embodiments, the method can reverse aging of a cell, tissue, or organ of a subject, thereby restoring viability of the cell, tissue, or organ.

In some embodiments, the method can promote axonal regeneration of a neuronal cell in a subject.

In some embodiments, the method can proliferate skin fibroblasts in a subject.

In some embodiments, the composition can promote proliferation of chondrocytes in a subject.

In some embodiments, the method can promote proliferation of muscle stem cells in a subject.

In some embodiments, the method can reverse aging of skin fibroblasts in a subject.

In some embodiments, the composition can reverse aging of chondrocytes.

In some embodiments, the method does not induce formation of teratoma or does not induce growth of tumor or formation of tumor.

In some embodiments, the method does not induce complete reprogramming.

In some embodiments, the method does not reprogram a cell to a pluripotent state, or the method does not reprogram a cell to an “induced pluripotent stem cell” or “iPSC”.

OCT4, SOX2, KLF4 and c-Myc are four “Yamanaka factors” which can reprogram a cell to a pluripotent state. However, inducing the expression of these four transcription factors in transgenic mice can lead to the formation of teratomas in vivo, as well as other acute toxicities.

The present invention unexpectedly found that, in the absence of OCT4 and c-Myc, any one of factors selected from the group consisting of SOX1, SOX2, SOX3, and GMNN, any one of factors selected from the group consisting of GATA3 and GATA6, and KLF4, that is, these three factors can achieve the reversal of aging cells.

The term “factor” in the present invention refers to a biologically active protein, or a nucleic acid encoding the same, such as DNA or RNA or a mixture thereof, which acts on a cell to alter transcription and, when expressed, transforms a senescent cell into a young cell. In some embodiments, the factor can be non-integrating, meaning it is provided to a recipient somatic cell in a form that does not result in integration of an exogenous DNA into the genome of a recipient cell.

The factor in the present invention can be a transcription factor, including but not limited to SOX1, SOX2, SOX3, GMNN, GATA3, GATA6, and Klf4.

### BRIEF DESCRIPTION OF THE DRAWINGS

FIG. 1 shows that the composition of the present invention can promote regeneration of neuronal cells.

FIG. 2 shows that the composition of the present invention can promote proliferation of fibroblasts in aging skin.

FIG. 3 shows that the composition of the present invention can promote proliferation of chondrocytes in mice.

FIG. 4 shows that the composition of the present invention can promote proliferation of muscle stem cells in aged mice.

FIG. 5 shows that the composition of the present invention can reverse aging of skin fibroblasts in aged mice.

FIG. 6 shows that the compositions of the present invention can reverse aging of chondrocytes.

### EMBODIMENTS

In the following examples and figures:
GATA3 is named A1, and GATA6 is named A2; SOX1 is named B1, and SOX2 is named B2, SOX3 is named B3, and GMNN is named B4; and Klf4 is named C.
SOX1, SOX2, SOX3, GMNN, GATA3, GATA6, and Klf4 used in the examples are all human SOX1, human SOX2, human SOX3, human GMNN, human GATA3, human GATA6, and human Klf4.

### Example 1. Compositions Promoting Axonal Regeneration of Neuronal Cells

The main purpose of this example is to select SH-SY5Y cells (human neuroblastoma cell line). differentiate them into neuronal cells and determine the regenerative effect of different compositions on neuronal cells.

The specific methods and results were as follows.

METHODS: One day after plating, the cells were induced to differentiate for 3 days, using EMEM/F12 medium (1:1) containing 2.5% FBS, 1×PS and 10 μM all-trans retinoic acid (ATRA, Stem cell Technologies, 72264) (Differentiation Medium 1), and then incubated for 3 days in EMEM/F12 medium (1:1) containing 1% FBS, 1×PS and 10 μM ATRA (Differentiation Medium 2). After the cells were then inoculated in cell culture plates coated with poly-D-lysine (ThermoFisher Scientific, A3890401) for 1 day and then the medium was replaced with serum-free B27 neuronal medium containing 1× Glutamax (Thermo Fisher Scientific, 35050061), PS and BDNF (Differentiation Medium 3). The cells were then cultured continually for at least 5 days to promote the differentiation and maturation of the cells. After differentiation was completed, the cells were treated with the composition mRNA for 5 days, and then vincristine (100 nM; Sigma, V8879) was added for 24 hours to induce nerve damage. The neurons were then washed once with PBS and fresh Differentiation Medium 3 was added to the plates to continue culturing for 9 days. The neuronal regeneration and maintenance were then statistically evaluated.

As shown in FIG. 1, the combination of A1/A2 with B1/B2/B3/B4 and C can promote an increase in neuronal area.

### Example 2. Compositions Promoting Proliferation of Skin Fibroblasts in Aged Mice

The main purpose of this example is to select skin cells of aged mice, and observe whether the proliferative capacity of the skin cells of aged mice is enhanced by administration of a drug.

The specific methods and results were as follows.

METHODS: 20-month-old aged mice were selected. After euthanizing the mice, the dorsal skin was shaved and washed with jodophor (povidone-jodine) and rinsed with cold 1×PBS, then excised the skin. Mice skin fibroblasts were separated with trypsin and collagenase. Fibroblasts were resuspended and cultured in DMEM glutamax medium. The cells were treated with the mRNA drug of the target composition, and after 6 days, the cells were collected to test whether the proliferative capacity of aging skin fibroblasts was enhanced.

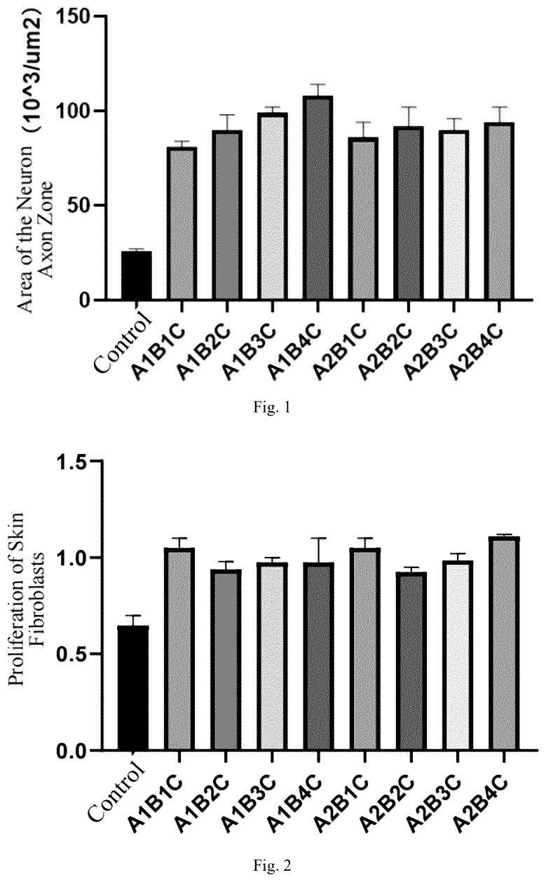

As shown in FIG. 2, the combination of A1/A2 with B1/B2/B3/B4 and C can promote the proliferation of aging skin fibroblasts.

### Example 3. Composition Promoting Proliferation of Chondrocytes in Mice

The main purpose of this example is to select aged mice, separate chondrocytes, and observe whether the chondrocytes exhibit an enhancer proliferation capacity.

The specific methods and results were as follows.

METHODS: 20-month-old aged mice were selected, the articular cartilage of mice was separated, and soft tissue was removed by gently stirring the tissue fragments, and the remaining cartilage fragments were transferred to a new petri dish and the articular cartilage was digested overnight. The collagenase solution was used to disperse cell aggregates by sequentially passing through the pipette and form a suspension of isolated cells, which were washed with PBS and resuspended in culture medium and centrifuged for incubation. After 6 days of administration, the proliferative capacity of chondrocytes was tested.

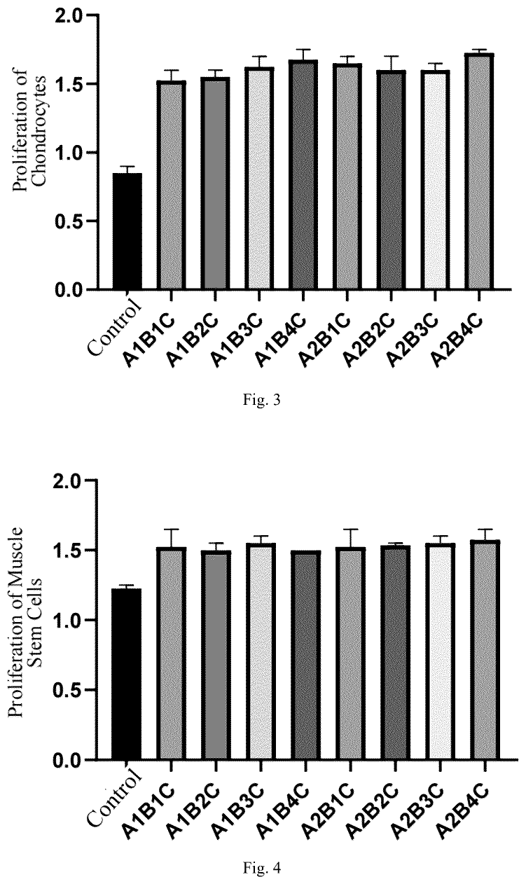

As shown in FIG. 3, the combination of A1/A2 with B1/B2/B3/B4 and C can promote the proliferation of aging chondrocytes.

### Example 4. Compositions Promoting Proliferation of Muscle Stem Cells in Aged Mice

The main purpose of this example is to select aged mice, separate muscle stem cells (MuSC), and observe whether the muscle stem cells exhibit an enhancer proliferation capacity after administration of the drug. The specific methods and results were as follows.

METHODS: 15-month-old aged mice were selected, the mice were euthanized by cervical dislocation, and the muscles were isolated, cut into small pieces, and incubated in DMEM medium (DMEM; Thermo Fisher Scientific) containing 0.2% type I collagenase (Sigma-Aldrich) at 37° C. for 2 hours. Single fibers were collected using a pipette tip and purified twice in DMEM containing 1 g/l glucose and supplemented with 10% horse serum (HS, ThermoFisher Scientific), 1% penicillin/streptomycin (ThermoFisher Scientific), and 0.5% chicken embryo extract (CEE, ThermoFisher Scientific). The single fibers were then transferred and the myofiber suspension was passed through a 40 μm filter using a syringe with a 21 G needle. The obtained stem cells were plated directly onto petri dishes coated with Matrigel Growth Factor (GFR) Basement Membrane Matrix (Corning) or petri dishes containing coverslips coated with Matrigel GFR Basement Membrane Matrix. Cells were proliferated in DMEM containing 1 g/l glucose and supplemented with 10% HS, 20% FBS, 1% ps, and 0.5% CEE and cultured at 37° C. with 5% CO₂. The medium was changed every two days. After treating the cells with different compositions for 6 days, the proliferative capacity of the cells was observed.

As shown in FIG. 4, the combination of A1/A2 with B1/B2/B3/B4 and C can promote the proliferation of aging muscle stem cells.

### Example 5. Compositions Reversing Aging of Skin Fibroblasts in Aged Mice

The main purpose of this example is to select skin cells of aged mice and observe whether the activity of the aging marker β-galactosidase in skin cells of aged mice changes by administering a drug. The specific methods and results were as follows.

The skin fibroblasts were separated from aged mouse and the cells were treated with the mRNA drug of the target composition, and after 6 days, the cells were fixed by adding 4% paraformaldehyde for 15 minutes, and then stained in situ using a β-galactosidase in situ staining kit (Beyotime). After staining was completed, DAPI was further added for nuclear staining in order to count the total number of cells. After that, the cells were observed under a fluorescence microscope and the ratio of positive cells was calculated (ratio of positive cell=number of stained positive cells/total number of cells×100%).

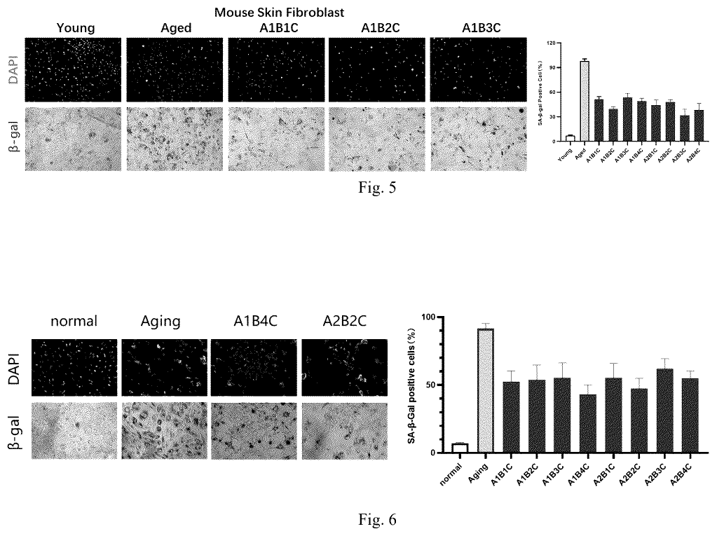

As shown in FIG. 5, the combination of A1/A2 with B1/B2/B3/B4 and C can significantly reverse the aging of skin fibroblasts in aged mice.

### Example 6. Compositions Reversing Aging of Chondrocytes

The main purpose of this example is to select chondrocytes, induce aging using 20 μM of etoposide, and then examine the change in the activity of the aging marker β-galactosidase in the cells by administering the composition. The specific methods and results were as follows.

Human chondrocyte cell line C28/12 was treated with 20 μM etoposide to induce aging, and 24 hours after induction, the cells were treated with the mRNA drug of the target combination, and after 6 days, the cells were fixed by adding 4% paraformaldehyde for 15 minutes, and then stained in situ using a β-galactosidase in situ staining kit (Beyotime). After staining was completed, DAPI was further added for nuclear staining in order to count the total number of cells. After that, the cells were observed under a fluorescence microscope and the ratio of positive cells was calculated (ratio of positive cell=number of stained positive cells/total number of cells×100%).

As shown in FIG. 6, the combination of A1/A2 with B1/B2/B3/B4 and C can significantly reverse aging of chondrocytes.

1. A composition for restoring viability of a cell, tissue or organ of a subject, wherein the composition comprises a factor selected from the group consisting of SOX1, SOX2, SOX3, and GMNN, a factor selected from the group consisting of GATA3 and GATA6, and a factor of Klf4.

2. The composition according to claim 1, wherein any one of factors of SOX1, SOX2, SOX3, GMNN, GATA3, GATA6, and Klf4 is a protein, a nucleic acid encoding the same or a mixture thereof,
preferably, any one of the factors of SOX1, SOX2, SOX3, GMNN, GATA3, GATA6, and Klf4 is a protein, a fusion protein with a transmembrane domain, or in a form of a nucleic acid encoding the same.

3. The composition according to claim 1, wherein any one of the factors of SOX1, SOX2, SOX3, GMNN, GATA3, GATA6, and Klf4 is in a form of an expression vector, and the expression vector is a plasmid vector, an episomal vector, a transposon, or a virus vector,
preferably, the virus vector is an adenovirus vector, an adeno-associated virus vector (AAV), a lentivirus vector, a Sendai virus vector, a cytomegalovirus vector, or a vaccinia virus vector, and
preferably, the factors are present on the same expression vector, or on multiple expression vectors.

4. The composition according to claim 1, wherein any one of the factors of SOX1, SOX2, SOX3, GMNN, GATA3, GATA6, and Klf4 is in a form of DNA or RNA,
preferably, any one of the factors is in a form of mRNA,
the factors are in tandem on a same mRNA molecule, or the factors of SOX1, SOX2, SOX3, GMNN, GATA3, GATA6, and Klf4 are in a form of a combination of different mRNA molecules, and
preferably, any one of the factors is a synthetic mRNA, and the synthetic mRNA encodes a wild type form, a mutant, or a genetically engineered form of any one of SOX1, SOX2, SOX3, GMNN, GATA3, GATA6, and Klf4.

5. The composition according to claim 1, wherein the subject is a mammal, and the mammal includes, but is not limited to, a human, a mouse, a rat, a cattle, a sheep, a horse, a canine, a cat, a pig, or a monkey,
wherein the cell, tissue, or organ of the subject is derived from, but not limited to, eye, ear, nose, mouth including gum and tooth root; bone, lung, breast, pancreas, stomach, esophagus; muscle, including cardiac muscle; liver, blood vessel; skin, including hair; heart, brain, neural tissue, kidney, testis, prostate, penis, cloaca, fin, ovary, or intestine.

6. The composition according to claim 1, wherein any one of the factors of SOX1, SOX2, SOX3, GMNN, GATA3, GATA6, and Klf4 is a human protein, or a non-human protein, or a protein having at least 80% identity to the amino acid sequence of the human protein or non-human protein, and maintaining the activity,
the non-human protein is a corresponding protein of mouse, rat, cattle, sheep, horse, canine, cat, pig, monkey, chicken, duck, goose, or bird; or
any one of the factors of SOX1, SOX2, SOX3, GMNN, GATA3, GATA6, and Klf4 is a nucleic acid encoding a human protein, or a nucleic acid encoding a non-human protein, or a nucleic acid having at least 80% identity to the sequence of the corresponding nucleic acid encoding the human protein or the non-human protein, and maintaining the activity, and
the nucleic acid is a nucleic acid encoding a corresponding protein of mouse, rat, cattle, sheep, horse, canine, cat, pig, monkey, chicken, duck, goose, and bird.

7. A method for restoring viability of a cell, tissue or organ of a subject, wherein the method comprises administering the composition according to claim 1 to a subject.

8. (canceled)

9. The method according to claim 7, wherein restoring viability of a cell, tissue or organ of a subject includes restoring transcriptional profile or epigenetic information lost in at least one cell, tissue, or organ due to aging, damage, disease, or any combination thereof.

10. The method according to claim 7, wherein restoring viability of a cell, tissue or organ of a subject includes restoring a function of the cell, increasing a potential of the cell, enhancing a viability of the cell, or increasing a replicative capacity or lifespan of the cell, or a combination thereof.

11. The method according to claim 7, wherein restoring viability of a cell, tissue or organ of a subject includes promoting axonal regeneration of a neuronal cell in a subject; proliferating skin fibroblasts in a subject; promoting proliferation of chondrocytes in a subject; promoting proliferation of muscle stem cells in a subject; reversing aging of skin fibroblasts in a subject; or reversing aging of chondrocytes.

12. The composition according to claim 1, wherein the subject suffers from, is suspected to suffer from, or is at a risk of suffering from:
a disease, condition, or symptom related to impaired viability of the cell, tissue or organ, or aging,
wherein the disease, condition, or symptom related to impaired viability of the cell, tissue or organ, or aging is a neurodegenerative disease, a cardiovascular disease, a metabolic disease, a musculoskeletal disease, an inflammatory disease, a symptom or disorder associated with skin, an ocular disease, or a disease associated with the reproductive system;
preferably, the neurodegenerative disease is stroke, Huntington's Disease, dementia, Alzheimer's Disease, or Parkinson's Disease;
preferably, the cardiovascular disease is hypertension, arrhythmia of coronary heart disease, heart disease, hypotension, heart failure, angina pectoris, arteriosclerosis, myocardial infarction, myocarditis, congestive heart failure, atrioventricular block, atherosclerosis, or myocardial infarction;
preferably, the metabolic disease is type I diabetes, type II diabetes, hyperglycemia, hyperinsulinemia, insulin resistance, obesity, or gout;
preferably, the musculoskeletal disease is muscular dystrophy, amyosthenia, myasthenia gravis, osteoporosis, osteoarthritis, osteochondrosis, osteochondritis, osteonecrosis, or osteopenia;
preferably, the inflammatory disease is systemic lupus erythematosus, polymyalgia rheumatica, gouty arthritis, degenerative arthritis, rheumatoid arthritis, inflammatory arthritis, Hashimoto's thyroiditis, inflammatory bowel disease, hepatitis, pneumonia, respiratory inflammation, or encephalitis;
preferably, the aging symptom or disorder associated with skin is wrinkle, senile plaque, seborrheic keratosis, tinea manus and pedis, urticaria, neurodermatitis, or pigmented nevus;
preferably, the ocular disease is macular degeneration, retinal degeneration, retinal detachment, diabetic retinopathy, glaucoma, optic atrophy, floaters, cataract, non-arteritic anterior ischemic optic neuropathy, chorioretinitis, retinitis pigmentosa, traumatic optic neuropathy, or compressive optic neuropathy; and
preferably, the disease associated with the reproductive system is erectile dysfunction or premature ovarian failure.

13. The method according to claim 7, wherein the subject suffers from, is suspected to suffer from, or is at a risk of suffering from:
a disease, condition, or symptom related to impaired viability of the cell, tissue or organ, or aging,
wherein the disease, condition, or symptom related to impaired viability of the cell, tissue or organ, or aging is a neurodegenerative disease, a cardiovascular disease, a metabolic disease, a musculoskeletal disease, an inflammatory disease, a symptom or disorder associated with skin, an ocular disease, or a disease associated with the reproductive system;
preferably, the neurodegenerative disease is stroke, Huntington's Disease, dementia, Alzheimer's Disease, or Parkinson's Disease;
preferably, the cardiovascular disease is hypertension, arrhythmia of coronary heart disease, heart disease, hypotension, heart failure, angina pectoris, arteriosclerosis, myocardial infarction, myocarditis, congestive heart failure, atrioventricular block, atherosclerosis, or myocardial infarction;
preferably, the metabolic disease is type I diabetes, type II diabetes, hyperglycemia, hyperinsulinemia, insulin resistance, obesity, or gout;
preferably, the musculoskeletal disease is muscular dystrophy, amyosthenia, myasthenia gravis, osteoporosis, osteoarthritis, osteochondrosis, osteochondritis, osteonecrosis, or osteopenia;
preferably, the inflammatory disease is systemic lupus erythematosus, polymyalgia rheumatica, gouty arthritis, degenerative arthritis, rheumatoid arthritis, inflammatory arthritis, Hashimoto's thyroiditis, inflammatory bowel disease, hepatitis, pneumonia, respiratory inflammation, or encephalitis;
preferably, the aging symptom or disorder associated with skin is wrinkle, senile plaque, seborrheic keratosis, tinea manus and pedis, urticaria, neurodermatitis, or pigmented nevus;
preferably, the ocular disease is macular degeneration, retinal degeneration, retinal detachment, diabetic retinopathy, glaucoma, optic atrophy, floaters, cataract, non-arteritic anterior ischemic optic neuropathy, chorioretinitis, retinitis pigmentosa, traumatic optic neuropathy, or compressive optic neuropathy; and
preferably, the disease associated with the reproductive system is erectile dysfunction or premature ovarian failure.
